(19) United States
(12) Patent Application Publication
(10) Pub. No.: US 2026/0076621 A1
(43) Pub. Date: Mar. 19, 2026

(54) ELECTRONIC DEVICE AND METHOD OF CONTROLLING SAME

(71) Applicant: Samsung Electronics Co., Ltd., Suwon-si (KR)

(72) Inventors: Jinhong MIN, Suwon-si (KR); Youngjae OH, Suwon-si (KR); Taehan JEON, Suwon-si (KR); Hyoungseon CHOI, Suwon-si (KR); Joonho KIM, Suwon-si (KR); Taeseon KIM, Suwon-si (KR); Seoyoung YOON, Suwon-si (KR)

(21) Appl. No.: 19/341,326

(22) Filed: Sep. 26, 2025

Related U.S. Application Data
(63) Continuation of application No. PCT/KR2024/002979, filed on Mar. 7, 2024.

(30) Foreign Application Priority Data
Mar. 27, 2023 (KR) 10-2023-0039945

(51) Int. Cl. A61B 5/00; A61B 5/1455

(52) U.S. Cl. CPC A61B 5/4875; A61B 5/1455; A61B 5/443; A61B 5/7282

(57) ABSTRACT

An electronic apparatus may include at least one sensor, a memory in which weights for different types of context information related to a user are stored, and one or more processors configured to: when first body water data of a user, corresponding to a first time period, is acquired on the basis of sensing data acquired through the at least one sensor, identify trend information of a plurality of pieces of valley data included in the acquired first body water data; identify dehydration criteria information of the user, on the basis of the trend information, current context information of the user, and the weight stored in the memory; and identify whether the user is dehydrated, on the basis of the body water data acquired through the at least one sensor and the identified dehydration criteria information of the user.

### CROSS-REFERENCE TO RELATED APPLICATIONS

This application is a continuation application of International Application No. PCT/KR2024/002979, filed on Mar. 7, 2024, in the Korean Intellectual Property Receiving Office, and claiming priority to Korean Patent Application No. 10-2023-0039945 filed Mar. 27, 2023, the disclosures of which are all hereby incorporated by reference herein in their entireties.

### TECHNICAL FIELD

Apparatuses and methods may relate to an electronic apparatus and/or a method of controlling the same, and for example, to an electronic apparatus that determines whether a user is dehydrated based on body water data and context information of the user, and/or a control of controlling the same.

### BACKGROUND

With the development of electronic technology, technological development for various types of electronic apparatuses is becoming active. Among these, electronic apparatuses that analyze body data of a user and provide information to the user, such as wearable devices, are also developing. For example, since excessive dehydration may affect exercise or cognitive abilities and even lead to death, information on the body water of the user may be used to notify the user of whether the user is dehydrated, thereby helping the user maintain his/her health.

Meanwhile, to determine whether the user is dehydrated, the information on the body water of the user is required, and setting a reference point for the body water of the user is crucial. In this case, a reference point for determining dehydration should be set by taking into account information (e.g., weight, age, body temperature, etc.) on physical conditions corresponding to each user.

### SUMMARY

In an example embodiment, an electronic apparatus may include at least one sensor, a memory in which weights for different types of context information related to a user are stored, and one or more processors, comprising processing circuitry, individually and/or collectively configured to, in response to first body water data of the user corresponding to a first time period is acquired based on sensing data acquired through the at least one sensor, identify trend information of a plurality of pieces of valley data included in the acquired first body water data.

The one or more processors may identify dehydration criteria information of the user, based on the trend information, current context information of the user, and the weight stored in the memory.

The one or more processors may identify whether the user is dehydrated, based on the body water data acquired through the at least one sensor and the identified dehydration criteria information of the user.

In an example embodiment, a method of controlling an electronic apparatus may include, in response to first body water data of a user corresponding to a first time period being acquired based on sensing data acquired through the at least one sensor, identifying trend information of a plurality of pieces of valley data included in the acquired first body water data.

The control method may include identifying the dehydration criteria information of the user based on the trend information, current context information of the user, and weights for different types of context information related to the user stored in memory.

The control method may include identifying whether the user is dehydrated, based on the body water data acquired through the at least one sensor and the identified dehydration criteria information of the user.

In an example embodiment, there may be provided a non-transitory computer-readable storage medium storing a computer command that, when executed by a processor of an electronic apparatus, causes the electronic apparatus to perform an operation, in which the operation may include, in response to first body water data of the user, corresponding to a first time period, being acquired based on sensing data acquired through the at least one sensor, identifying trend information of a plurality of pieces of valley data included in the acquired first body water data.

The operation may include identifying the dehydration criteria information of the user based on the trend information, current context information of the user, and weights for different types of context information related to the user stored in memory.

The operation may include identifying whether the user is dehydrated, based on the body water data acquired through the at least one sensor and the identified dehydration criteria information of the user.

### BRIEF DESCRIPTION OF DRAWINGS

FIG. 1 is a diagram for schematically describing a method of controlling an electronic apparatus according to an example embodiment.

FIG. 2 is a block diagram illustrating a configuration of the electronic apparatus according to an example embodiment.

FIG. 3 is a flowchart for describing the method of controlling an electronic apparatus according to an example embodiment.

FIG. 4 is a diagram for describing body water data, trend information, and dehydration criteria information according to an example embodiment.

FIG. 5 is a diagram for describing a method of updating trend information and dehydration criteria information according to an example embodiment.

FIG. 6 is a diagram for describing a method of providing notification information according to an example embodiment.

FIGS. 7A and 7B are diagrams for describing a method of identifying trend information according to an example embodiment.

FIGS. 8A and 8B are diagrams for describing a method of identifying a weight according to an example embodiment.

FIG. 9 is a diagram for describing a method of identifying a weight according to an example embodiment.

FIGS. 10A and 10B are diagrams for describing a method of updating dehydration criteria information according to an example embodiment.

FIG. 11 is a diagram for describing a method of updating dehydration criteria information according to an example embodiment.

FIG. 12 is a block diagram illustrating a detailed configuration of an electronic apparatus according to an example embodiment.

### DETAILED DESCRIPTION

Hereinafter, the present disclosure will be described in detail with reference to the accompanying drawings.

After terms used in the present specification are briefly described, the present disclosure will be described in detail.

General terms that are currently widely used were selected as terms used in embodiments of the present disclosure in consideration of functions in the present disclosure, but may be changed depending on the intention of those skilled in the art or a judicial precedent, the emergence of a new technique, and the like. In addition, in a specific case, terms arbitrarily chosen by an applicant may exist. In this case, the meaning of such terms will be mentioned in detail in a corresponding description portion of the present disclosure. Therefore, the terms used in the present disclosure should be defined on the basis of the meaning of the terms and the contents throughout the present disclosure rather than simple names of the terms.

In the disclosure, an expression “have,” “may have,” “include,” “may include,” or the like, indicates existence of a corresponding feature (for example, a numerical value, a function, an operation, a component such as a part, or the like), and does not exclude existence of an additional feature.

An expression “at least one of A and/or B” is to be understood to represent “A” or “B” or “any one of A and B.”

Expressions “first,” “second,” “1st” or “2nd” or the like, used in the present specification may indicate various components regardless of a sequence and/or importance of the components, will be used only in order to distinguish one component from the other components, and do not limit the corresponding components.

When it is mentioned that any component (for example: a first component) is (operatively or communicatively) coupled with/to or is connected to another component (for example: a second component), it is to be understood that any component is directly coupled to another component or may be coupled to another component through the other component (for example: a third component).

Singular expressions are intended to include plural expressions unless the context clearly indicates otherwise. It will be further understood that the terms “comprises” or “have” used in this specification, specify the presence of stated features, steps, operations, components, parts mentioned in this specification, or a combination thereof, but do not preclude the presence or addition of one or more other features, numerals, steps, operations, components, parts, or a combination thereof.

In the disclosure, a “module” or a “˜er/or” may perform at least one function or operation, and be implemented by hardware or software or be implemented by a combination of hardware and software. In addition, a plurality of “modules” or a plurality of “˜ers/˜ors” may be integrated in at least one module and be implemented by at least one processor (not illustrated) except for a “module” or a “˜er/or” that needs to be implemented by specific hardware.

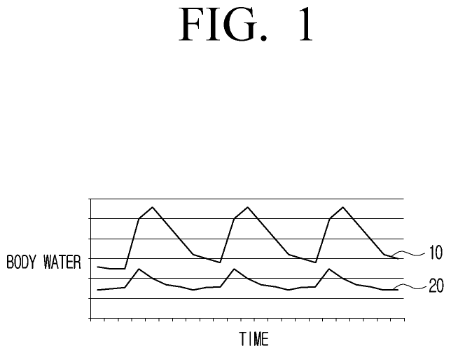

FIG. 1 is a diagram for schematically describing a method of controlling an electronic apparatus according to an embodiment.

According to an embodiment, an electronic apparatus may acquire body water data of a user, and acquire trend information of the body water data based on the acquired body water data. Generally, the amount of body water in animals, including humans, varies depending on various factors. The change in body water over time may be represented as a curved graph having at least one peak value and a valley value, as illustrated in FIG. 1.

Meanwhile, the amount of body water of a user may vary depending on user's physical information, such as body temperature, age, medication information, and drinking status, as well as environmental information, such as the ambient temperature and humidity of the user. For example, even for a first user and a second user in the same environment, body water data 10 corresponding to the first user and body water data 20 corresponding to the second user may have different values. Therefore, even when measuring the body water data of the user using the same electronic apparatus, it is necessary to be able to determine whether the user is dehydrated by considering the characteristics corresponding to each user.

Hereinafter, various embodiments of determining whether the user is dehydrated will be described using different types of user context information and weights corresponding to each type of context information.

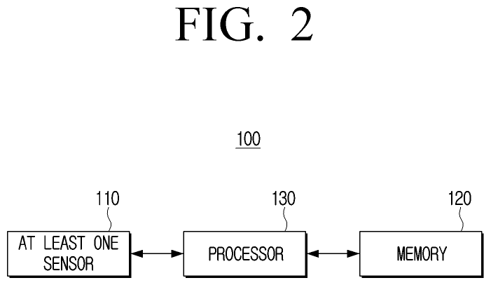

FIG. 2 is a block diagram illustrating a configuration of the electronic apparatus according to an embodiment.

Referring to FIG. 2, the electronic apparatus 100 may include at least one sensor 110, a memory 120, and one or more processors 130.

The electronic apparatus 100 may be implemented as a wearable device according to an embodiment. The wearable device is a device implemented in a form in which the wearable device is worn by a user or attached to or inserted into skin, and may be a smart watch, a smart band, smart glasses, a smart ring, a head mounted display (HMD), etc. However, the present disclosure is not limited thereto, and any type of electronic apparatus may be used as long as the electronic apparatus is worn by the user or attached to or inserted into the skin.

Meanwhile, the electronic apparatus 100 may communicate with external devices and external servers in various ways. According to an embodiment, communication modules for communication with external devices and external servers may be implemented identically. For example, the electronic apparatus 100 may communicate with the external devices using a Bluetooth module, and may also communicate with the external servers using the Bluetooth module.

At least one sensor 110 (hereinafter referred to as a sensor) may include a plurality of sensors of various types. The sensor 110 may measure a physical quantity or detect an operating state of the electronic apparatus 100 and convert the measured or sensed information into an electrical signal. The sensor 110 may include a camera, and the camera may include a lens for focusing visible light and other optical signals received after being reflected by an object into an image sensor, and an image sensor capable of detecting visible light and other optical signals. Here, the image sensor may include a 2D pixel array divided into a plurality of pixels.

According to an embodiment, at least one sensor 110 may include at least one of an impedance sensor, a photoplethysmography (PPG) sensor, an electrodemical activity (EDA) sensor, a temperature sensor, or an acceleration sensor. The PPG sensor is a sensor that identifies a heart rate activity state by measuring the amount of blood flowing in blood vessels using optical properties of skin. The EDA sensor is a sensor that provides information on an autonomic nervous system of a human body by combining with a human heart rate, a respiration rate, blood pressure, etc. The processor 130 may acquire the body water data of the user through sensing information received from at least one of the impedance sensor, the photoplethysmography (PPG) sensor, the electrodemical activity (EDA) sensor, or the temperature sensor using a preset algorithm.

The memory 120 may store data necessary for various embodiments. The memory 120 may be implemented in a form of a memory embedded in the electronic apparatus 100 or a form of a memory attachable to and detachable from the electronic apparatus 100, depending on a data storing purpose. For example, data for driving the electronic apparatus 100 may be stored in the memory embedded in the electronic apparatus 100, and data for an extension function of the electronic apparatus 100 may be stored in the memory attachable to and detachable from the electronic apparatus 100.

Meanwhile, the memory embedded in the electronic apparatus 100 may include at least one of, for example, a volatile memory (for example, a dynamic random access memory (DRAM), a static RAM (SRAM), a synchronous dynamic RAM (SDRAM), or the like), a non-volatile memory (for example, a one time programmable read only memory (OTPROM), a programmable ROM (PROM), an erasable and programmable ROM (EPROM), an electrically erasable and programmable ROM (EEPROM), a mask ROM, a flash ROM, or the like), a flash memory (for example, a NAND flash, a NOR flash, or the like), a hard drive, and a solid state drive (SSD)). In addition, the memory attachable to and detachable from the electronic apparatus 100 may be implemented in the form of the memory card (e.g., compact flash (CF), secure digital (SD), micro secure digital (Micro-SD), mini secure digital (Mini-SD), extreme digital (xD), multi-media card (MMC), etc.), external memory (e.g., USB memory) connectable to a USB port, and the like.

According to an embodiment, the memory 120 may store weights for different types of context information related to the user. Here, the context information related to the user refers to at least one of information related to the user's body or information on the environment surrounding the user wearing the electronic apparatus 100.

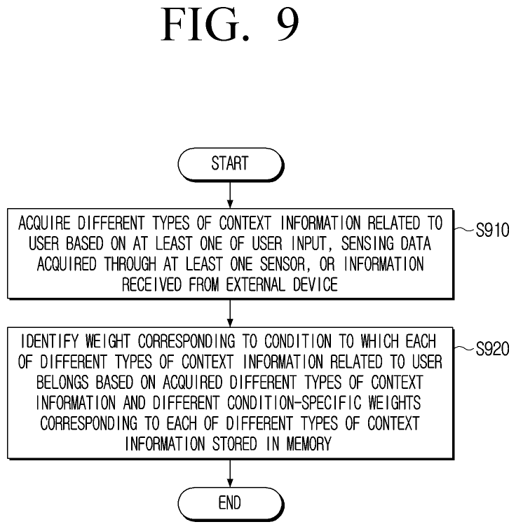

According to one example, the context information related to the user may include at least one of the user's weight, the user's height, age, body temperature, exercise frequency, drinking status, medication information, the ambient temperature of the electronic apparatus 100, and the ambient humidity of the electronic apparatus 100. Meanwhile, according to one example, the memory 120 may store at least one weight value corresponding to each of the different types of context information. For example, different condition-specific weights corresponding to each of the context information may be stored in the memory 120, and the processor 130 may identify at least one weight based on the context information. This will be described in detail with reference to FIG. 9.

One or more processors 130 (hereinafter referred to as processors) are electrically connected, directly or indirectly, to at least one sensor 110 and the memory 120 to control the overall operation of the electronic apparatus 100. The processor 130 may be composed of one or a plurality of processors. Specifically, the processor 130 may perform an operation of the electronic apparatus 100 according to various embodiments of the present disclosure by executing at least one instruction stored in the memory 120.

According to an embodiment, the processor 130 may be implemented by a digital signal processor (DSP), a microprocessor, a graphics processing unit (GPU), an artificial intelligence (AI) processor, a neural processing unit (NPU), or a time controller (TCON) that processes a digital image signal. However, the processor 110 is not limited thereto, and may include one or more of a central processing unit (CPU), a micro controller unit (MCU), a micro processing unit (MPU), a controller, an application processor (AP), a communication processor (CP), and an ARM processor, or may be defined by these terms. In addition, the processor 130 may be implemented by a system-on-chip (SoC) or a large scale integration (LSI) in which a processing algorithm is embedded, or may be implemented in the form of an application specific integrated circuit (ASIC) and a field programmable gate array (FPGA).

According to an embodiment, the processor 130 may acquire the body water data of the user based on the sensing data acquired through at least one sensor 110. The body water refers to the amount of water contained within a body, including tissue, blood, or bones of an animal. The body water data may include information on a body water value of a user at a specific time. According to one example, the body water data may include information on a graph corresponding to the change in the body water value over time.

According to an embodiment, the processor 130 may identify trend information of a plurality of pieces of valley data included in the body water data. Generally, the amount of body water of animals, including humans, varies depending on various factors, and the change in the body water value over time may be expressed as a curved graph having at least one peak value and one valley value. According to one example, the body water data may include peak data corresponding to a peak value included in a graph corresponding to the body water value of the user, and valley data corresponding to a valley value.

Meanwhile, the trend information of the valley data refers to information on a moving average for a valley data set corresponding to the plurality of pieces of valley data. Here, the moving average refers to an average calculated continuously over time for a subset of data of a preset size among a plurality of pieces of data. When the plurality of pieces of valley data are acquired over a preset time period, the processor 130 may acquire moving average information (or a moving average line) for the plurality of pieces of valley data over time.

For example, calculating an average for a subset of data of a preset size (5 pieces of data) is considered. When there are first to tenth data points corresponding to time points t1 to t10, respectively, the moving average information may be calculated using a first average value (average value of data corresponding to time points t1 to t5), a second average value (average value of data corresponding to time points t2 to t6), . . . , and a sixth average value (average value of data corresponding to time points t6 to t10).

However, the present disclosure is not limited thereto. According to one example, it goes without saying that the processor 130 may acquire the trend information corresponding to the plurality of pieces of valley data using the preset algorithm.

According to one example, when first body water data of a user corresponding to a first time period is acquired based on sensing data acquired through at least one sensor 110, the processor 130 may identify the trend information of the plurality of pieces of valley data included in the acquired first body water data. For example, the first time period may be a time period from the time the power is turned on to the time when 24 hours have elapsed, and the processor 130 may acquire the first body water data, which is body water data of a user for 24 hours from the time the user turns on the power of the electronic apparatus 100, based on the sensing data acquired through at least one sensor 110. The processor 130 may identify the plurality of pieces of valley data included in the acquired first body water data and, based on the plurality of pieces of identified valley data, identify the trend information of the plurality of pieces of valley data included in the first body water data.

According to an embodiment, the processor 130 may identify dehydration criteria information of a user. Here, the dehydration criteria information of the user refers to information on a reference value for determining whether the user is dehydrated based on the body water data of the user. Since the body water value of the user changes over time, the dehydration criteria information of the user needs to be updated over time to reflect the change in the body water value.

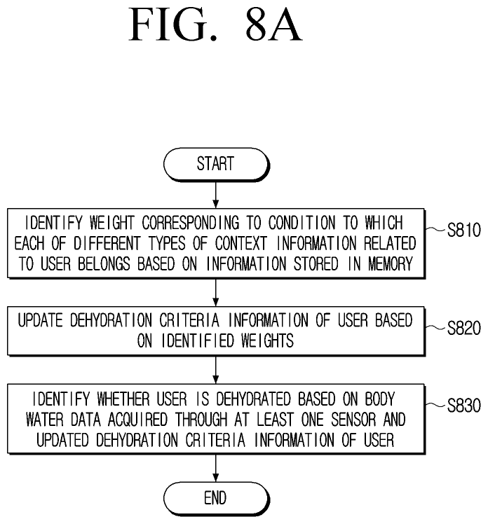

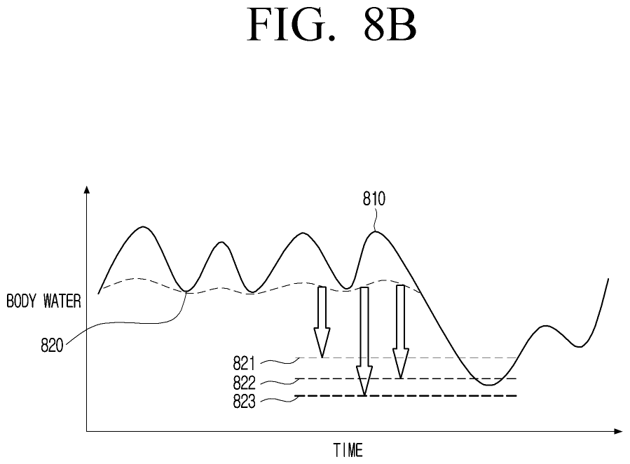

According to one example, the processor 130 may identify the dehydration criteria information of the user based on the identified trend information, the current context information of the user, and the weights stored in the memory 120. For example, when the first body water data of the user is acquired during a first time period corresponding to the time point at which 24 hours have elapsed from the time point at which the user turns on the electronic apparatus 100, the processor 130 may identify the trend information of the plurality of pieces of valley data included in the first body water data. When the current context information of the user is acquired, the processor 130 may identify the weight based on the acquired current context information of the user and the information stored in the memory 120. The processor 130 may identify the dehydration criteria information of the user by multiplying the identified trend information by the identified weight. Meanwhile, a specific method of identifying a weight will be described in detail with reference to FIGS. 8A and 8B.

According to an embodiment, the processor 130 may identify whether the user is dehydrated based on the body water data acquired through at least one sensor 110 and the identified dehydration criteria information of the user.

According to one example, the processor 130 may acquire the body water data of the user in real time based on the sensing data acquired through at least one sensor 110, and compare the acquired real-time body water data of the user with the identified dehydration criteria information of the user to determine whether the user is dehydrated. For example, when the real-time body water value of the user is identified as being less than the dehydration criteria information, the processor 130 may identify that the user is currently in the dehydrated state.

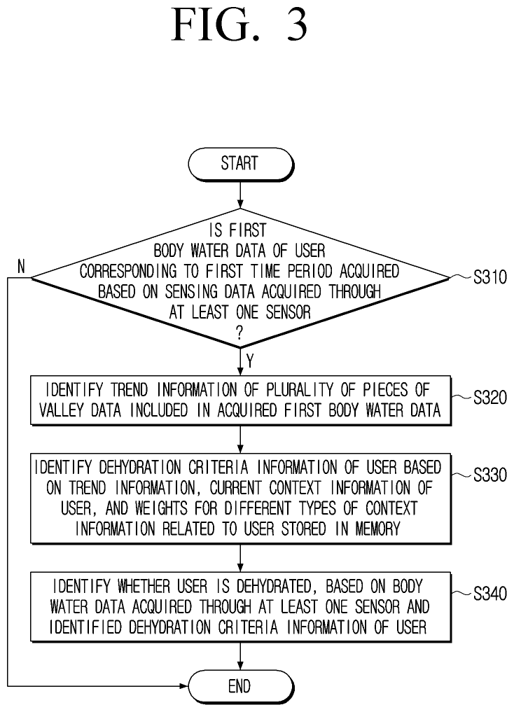

FIG. 3 is a flowchart for describing the method of controlling an electronic apparatus according to an embodiment.

Referring to FIG. 3, according to an embodiment, the control method may identify whether the first body water data of the user corresponding to the first time period is acquired based on the sensing data acquired through at least one sensor 110 (S310). According to one example, the processor 130 may acquire the first body water data, which is the body water data of the user for 24 hours from the time point at which the user turns on the electronic apparatus, based on the sensing data acquired through at least one sensor 110.

Subsequently, according to an embodiment, when the first body water data of the user corresponding to the first time period is acquired (S310: Y), the control method may identify the trend information of the plurality of pieces of valley data included in the acquired first body water data (S320). According to one example, the processor 130 may identify the plurality of pieces of valley data included in the acquired first body water data, and identify the trend information of the plurality of pieces of valley data included in the first body water data using the plurality of pieces of valley data.

Next, according to an embodiment, the control method may identify the dehydration criteria information of the user based on the trend information, the current context information of the user, and the weights for the different types of context information related to the user stored in the memory 120 (S330). According to one example, when the current context information of the user is acquired, the processor 130 may identify the weights based on the acquired current context information of the user and the information stored in the memory 120. Subsequently, according to an embodiment, the processor 130 may identify the dehydration criteria information of the user by multiplying the identified trend information by the identified weights.

Next, according to an embodiment, the control method may identify whether the user is dehydrated based on the body water data acquired through at least one sensor 110 and the identified dehydration criteria information of the user (S340). According to one example, the processor 130 may compare the acquired real-time body water data of the user with the identified dehydration criteria information of the user to determine whether the user is dehydrated.

According to the above-described example, the electronic apparatus 100 may acquire the dehydration criteria information of the user by using the body water data of the user, the different types of context information of the user, and the weights corresponding to each of the different types of context information, and provide the information related to whether the user is dehydrated based on the acquired dehydration criteria information. The electronic apparatus 100 may determine whether the user is dehydrated by reflecting the context information of the user, and thus may provide the information related to whether the user is dehydrated by taking into account the individual characteristics of the user.

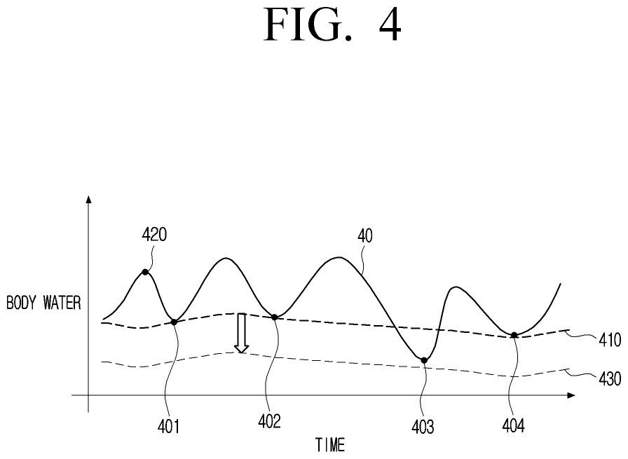

FIG. 4 is a diagram for describing body water data, trend information, and dehydration criteria information according to an embodiment.

Referring to FIG. 4, according to an embodiment, when body water data 40 of the user is acquired based on the sensing data acquired through at least one sensor 110, the processor 130 may acquire dehydration criteria information 430 of the user using the acquired body water data 40. Here, the body water data may be graph information on the body water value of the user over time.

According to one example, the body water data of the user 40 may include a plurality of pieces of peak data 420 and a plurality of pieces of valley data 401 to 404. The processor 130 may identify the trend information of the plurality of pieces of valley data included in the body water data based on the acquired body water data. For example, the processor 130 may identify the plurality of pieces of valley data 401 to 404 included in the first body water data of the user during the first time period and identify a moving average 410 for the identified valley data. The processor 130 may identify the acquired moving average 410 as the trend information of the first body water data.

In one example, when the trend information of the first body water data is identified, the processor 130 may acquire the dehydration criteria information 430 for the user by multiply the identified trend information by the weight.

For example, the processor 130 may acquire different types of context information, including the age, weight, drinking status, and medication status of the user, and identify a weight corresponding to the context information of the user among the weights corresponding to each of the different types of context the information stored in the memory 120. The processor 130 may acquire the dehydration criteria information of the user 430 by multiplying the identified trend information by the identified weight. This will be described in detail with reference to FIGS. 8A and 8B.

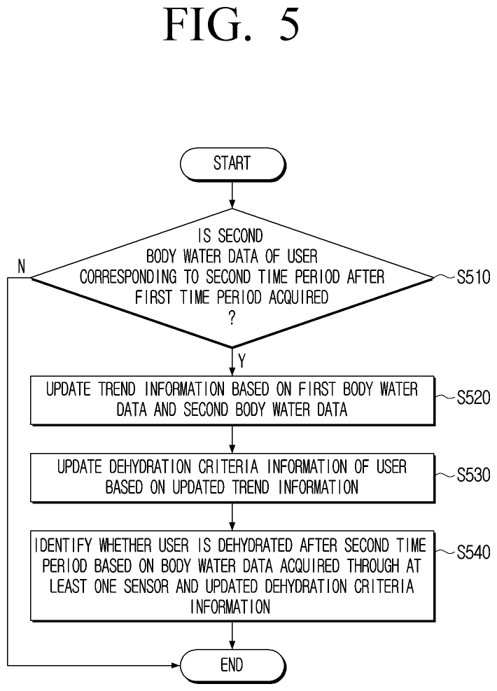

FIG. 5 is a diagram for describing a method of updating trend information and dehydration criteria information according to an embodiment.

According to an embodiment, the control method may first determine whether the second body water data of the user corresponding to the second time period after the first time period is acquired (S510). According to one example, the processor 130 may acquire the second body water data corresponding to the second time period, which is the time period corresponding to the time point when the preset time has elapsed from the time point at which the first time period has elapsed, after the first time period has elapsed. However, the present disclosure is not limited thereto, and according to one example, the processor 130 may also acquire the body water data of the user after the time point at which the first time period has elapsed in real time.

Next, according to an embodiment, when the control method identifies that the second body water data of the user has been acquired (S510: Y), the control method may update the trend information based on the first body water data and the second body water data (S520). According to one example, when the second body water data corresponding to the second time period is acquired, the processor 130 may identify the valley data included in the first body water data and the second body water data, and use the identified valley data to identify the updated trend information.

For example, it is assumed that trend information corresponding to the first body water data has been identified. When the single valley data is identified during the second time period from the time point at which the first time period has elapsed, the processor 130 may use the identified valley data and the identified trend information to identify the updated trend information. In other words, the processor 130 may use the first body water data and the valley data identified during the second time period to identify the moving average for the valley data, and identify the moving average as the updated trend information.

In this way, the processor 130 may update the trend information in real time using the valley data included in the body water data acquired in real time after the first time period has elapsed.

Subsequently, according to an embodiment, the control method may update the dehydration criteria information of the user based on the updated trend information (S530). According to one example, the processor 130 may update the dehydration criteria information of the user by multiplying the updated trend information by the weight. For example, the processor 130 may identify the weight based on the context information of the user and the information stored in the memory 120, and acquire the updated dehydration criteria information by multiplying the identified weight by the updated trend information.

Next, according to an embodiment, the control method may identify whether the user is dehydrated after the second time period based on the body water data acquired through at least one sensor 110 and the updated dehydration criteria information (S540). According to an embodiment, the processor 130 may identify whether the user is dehydrated after the first time period using the body water data acquired in real time and the updated dehydration criteria information.

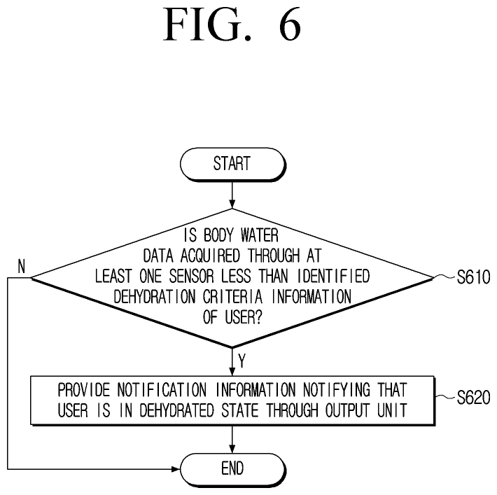

FIG. 6 is a diagram for describing a method of providing notification information according to an embodiment.

Referring to FIG. 6, according to an embodiment, the control method may first identify whether the body water data acquired through at least one sensor 110 is less than the identified dehydration criteria information of the user (S610). According to one example, the processor 130 may acquire the body water data of the user in real time through at least one sensor 110 and compare the acquired real-time body water data with the dehydration criteria information of the user to determine whether the acquired body water data is less than the identified dehydration criteria information of the user.

Subsequently, according to an embodiment, when it is identified that the acquired body water data is below the identified dehydration criteria information of the user (S610: Y), the control method may provide the notification information indicating that the user is in the dehydrated state through the output unit (not illustrated). According to one example, the notification information may include at least one of information indicating that the user is currently in the dehydrated state or information providing guidance for the user to overcome the dehydration state.

Meanwhile, according to one example, the output unit (not illustrated) may be implemented as a display (not illustrated) or a speaker (not illustrated), but is not limited thereto. For example, the processor 130 may provide a graphical user interface (UI) including the notification information via a display (not illustrated) or a sound user interface (UI) corresponding to the notification information via a speaker (not illustrated).

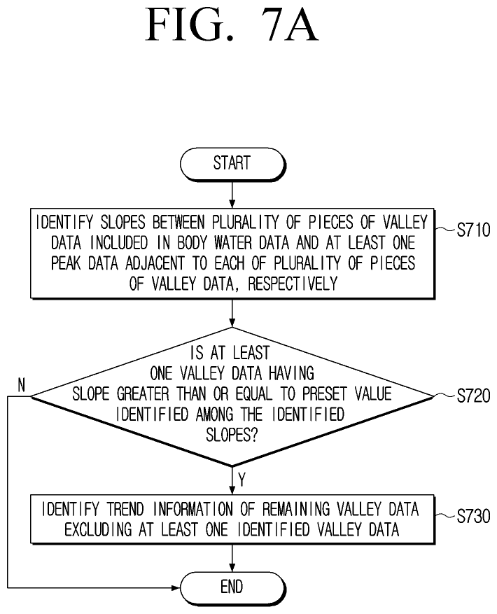

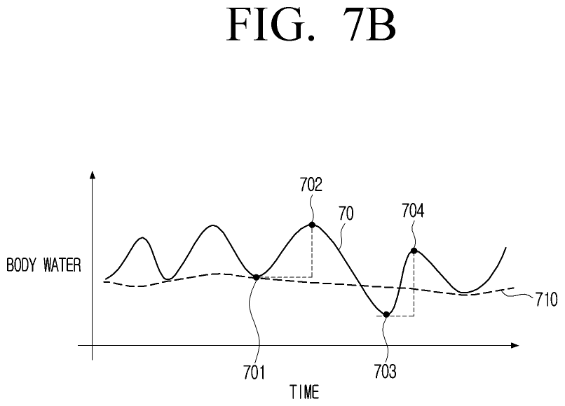

FIGS. 7A and 7B are diagrams for describing a method of identifying trend information according to an embodiment.

Referring to FIG. 7A, according to an embodiment, the control method may first identify the plurality of pieces of valley data included in the body water data and a slope between each of the plurality of pieces of valley data and at least one adjacent peak data (S710).

According to an example, as illustrated in FIG. 7B, the processor 130 may identify the slope between each of the plurality of pieces of valley data included in the body water data of the user and each of the valley data and at least one adjacent peak data.

For example, the processor 130 may identify a first slope between first valley data 703 and first peak data 702 adjacent to the first valley data 703, and a second slope between the first valley data 703 and the second peak data 704 adjacent to the first valley data 703, respectively.

Referring back to FIG. 7A, according to an embodiment, the control method may identify whether at least one valley data has a slope greater than or equal to a preset value among the identified slopes (S720).

According to one example, as illustrated in FIG. 7B, when the first slope between the first valley data 703 and the first peak data 702 adjacent thereto and the second slope between the first valley data 703 and the second peak data 704 adjacent thereto are identified, it may be identified whether at least one of the identified first and second slopes has a slope greater than or equal to the preset value.

Referring back to FIG. 7A, according to an embodiment, when there is at least one valley data having a slope greater than or equal to the preset value (S720: Y), the control method may identify the trend information of the remaining valley data excluding the at least one identified valley data (S730).

According to one example, the processor 130 may identify whether there is at least one valley data having a slope greater than or equal to the preset value among the identified first slope and second slope. When at least one valley data having a slope greater than or equal to the preset value is identified among the identified first slope and second slope, the processor 130 may identify the trend information using the remaining valley data, excluding the identified at least one valley data.

Generally, the slope between valley data (e.g., 703) that deviates from the trend information 710 and the peak data adjacent thereto has a relatively larger value than the slope between the valley data (e.g., 701) that does not deviate from the trend information 710 and the peak data adjacent thereto. By identifying the trend information while excluding data belonging to outlier data, such as the valley data (e.g., 703) that deviates from trend information 710, the accurate dehydration criteria information may be identified, thereby providing accurate alarms to the user.

FIGS. 8A and 8B are diagrams for describing a method of identifying a weight according to an embodiment.

Referring to FIG. 8A, according to an embodiment, the control method first identifies weights corresponding to conditions for each type of context information associated with a user based on the information stored in the memory 120 (S810).

According to one example, the memory 120 may store not only weights for each type of context information associated with a user, but also different condition-specific weights corresponding to each type of context information. According to one example, the processor 130 may update the weights for each type of context information based on the current user context information and the different condition-specific weights corresponding to each type of context information.

For example, in the case of the context information of the weight type, the condition in which the user's weight is identified as being greater than or equal to the preset value and the condition in which the user's weight is identified as being less than the preset value may be included. In this case, the weight value corresponding to the case in which the user's weight is greater than or equal to the preset value and the weight value corresponding to the case in which the user's weight is less than the preset value may be stored in the memory 120. For example, when it is identified that the user's weight is greater than or equal to the preset value, the corresponding weight value may be a value less than 1. When the user's weight increases, the body water value of the user generally increases, making it less likely for the user to reach the dehydrated state compared to when the user's weight decreases. Accordingly, by lowering the reference value for determining the dehydration, it is possible to avoid providing the user with indiscriminate notification information.

Alternatively, for example, the case of the context information of the type ‘ambient temperature of the electronic apparatus 100’ is considered. When the ambient temperature of the electronic apparatus 100 rises, the body water value of the user decreases to a relatively large value compared to when the ambient temperature of the electronic apparatus 100 decreases, so the user may easily reach the dehydrated state. In this case, the weight corresponding to the condition in which the ambient temperature of the electronic apparatus 100 is identified as being higher than the preset value may be less than 1, and the weight corresponding to the condition in which the ambient temperature of the electronic apparatus 100 is identified as being lower than a preset value may be greater than 1.

Meanwhile, the processor 130 may acquire the user context information based on at least one of the user input, the sensing data acquired through at least one sensor 110, or the information received from the external device (not illustrated), and may identify the weights corresponding to the conditions to which each type of context information belongs based on this information.

Subsequently, according to an embodiment, the control method may update the dehydration criteria information of the user based on the identified weights (S820). According to one example, the processor 130 may update the dehydration criteria information of the user using the following Equation (1):

$$\text{?} \qquad [ Equation 1 ]$$

$$\text{?} \text{indicates text missing or illegible when filed}$$

Here, N represents the number of context information types. An initial TBW threshold represents initial dehydration criteria information of the user. Weight_N represents the identified weights corresponding to each of the different type of context information. A corrected TBW threshold represents the updated dehydration criteria information.

According to one example, the processor 130 may acquire the user context information based on at least one of the user input, the sensing data acquired through at least one sensor 110, or the information received from the external device (not illustrated). For example, the processor 130 may identify the user context information corresponding to the time point (or the preset time period from the time point at which the device is turned on) at which the electronic apparatus 100 is turned on as the initial context information.

According to one example, the processor 130 may update the weights for each type of context information based on the current user context information and the different condition-specific weights corresponding to each type of context information. The processor 130 may identify the dehydration criteria information reflecting the updated weight using the above Equation 1.

Meanwhile, according to one example, the processor 130 may also identify different conditions corresponding to different types of context information based on the initial context information. For example, in the case of the context information of the weight type, the processor 130 may identify the condition in which the user's weight is identified as being greater than or equal to the user's weight value included in the initial context information, and the condition in which the user's weight is identified as being less than or equal to the user's weight value included in the initial context information, as the condition corresponding to the weight. However, the present disclosure is not limited thereto, and according to one example, different conditions corresponding to different types of context information may be pre-stored in the memory 120.

Next, according to an embodiment, the control method may identify whether the user is dehydrated based on the body water data acquired through at least one sensor 110 and the updated dehydration criteria information of the user (S830). According to one example, when the dehydration criteria information with the updated weight reflected based on the current context information of the user is acquired, the processor 130 may identify whether the user is dehydrated based on the acquired dehydration criteria information and the acquired body water data.

Referring to FIG. 8B, according to an embodiment, when trend information 820 is identified based on body water data 810 of a user, the processor 130 may identify the dehydration criteria information of the user based on the identified trend information, the current context information of the user, and the weights stored in the memory 120.

In this case, according to an embodiment, the processor 130 may acquire the updated dehydration criteria information using the different condition-specific weights corresponding to the different types of context information.

According to one example, the processor 130 may first identify first dehydration criteria information 822 using the context information of the user and the weights for the different types of context information related to the user.

Thereafter, when the current context information of the user is acquired, the processor 130 may identify the weights corresponding to the conditions to which each type of context information related to the user belongs based on the acquired current context information and the information stored in the memory 120. For example, as it is identified that the user's weight is greater than or equal to the preset value based on the acquired current context information, when the “condition in which the user's weight is identified as greater than or equal to the preset value” is established, the processor 130 may identify the weight corresponding to the established condition. The weight corresponding to the established condition may be a value less than 1.

Thereafter, when the weight corresponding to the condition is identified, the processor 130 may acquire the updated dehydration criteria information based on the identified weight. For example, when the weight corresponding to the established condition is less than 1, the updated dehydration criteria information may be information 823 having a lower reference value than the dehydration criteria information 822 prior to the update. Alternatively, when the weight corresponding to the established condition is greater than 1, the updated dehydration criteria information may be information 821 having a higher reference value than the dehydration criteria information 822 prior to the update.

According to the above-described example, the electronic apparatus 100 may update the dehydration criteria information by reflecting the current context information of the user, and the user convenience may be increased by providing the updated dehydration criteria information to the user.

FIG. 9 is a diagram for describing a method of identifying a weight according to an embodiment.

Referring to FIG. 9, according to an embodiment, the control method may acquire the different types of contextual information related to the user based on at least one of the user input, the sensing data acquired through at least one sensor 110, or the information received from an external device (S910).

According to an example, the different types of contextual information related to the user may include at least one of the user's weight, height, age, body temperature, exercise frequency, drinking status, medication information, the ambient temperature of the electronic apparatus 100, and the ambient humidity of the electronic apparatus 100. The processor 130 may acquire physical information on the user, such as the user's weight, height, age, and body temperature, through at least one sensor 110. Alternatively, when at least one sensor 110 is implemented as a temperature sensor or a humidity sensor, the processor 130 may acquire at least one of the ambient temperature of the electronic apparatus 100 and the ambient humidity of the electronic apparatus 100 through the sensing data acquired through at least one sensor 110.

Meanwhile, according to an example, the processor 130 may acquire at least one of the user's weight, height, age, body temperature, exercise frequency, drinking status, and medication information through the user input. For example, the processor 130 may receive the user input regarding at least one of the user's weight, height, age, body temperature, exercise frequency, drinking status, and medication information through the user interface (not illustrated). Alternatively, according to an example, the processor 130 may acquire the user's weight, height, age, body temperature, exercise frequency, drinking status, medication information, the ambient temperature of the electronic apparatus 100, and the ambient humidity of the electronic apparatus 100 from the external device (not illustrated) through the communication interface comprising interface circuitry (not illustrated).

Next, according to an embodiment, the control method may identify the weight corresponding to the condition to which each type of context information belongs, based on the different condition-specific weights corresponding to each of the acquired different types of context information and each of the different types of context information stored in the memory 120 (S920). According to one example, when the different types of context information of the user are acquired, the processor 130 may identify the condition that is satisfied among the different conditions corresponding to each of the context information based on the acquired context information, and identify the weight corresponding to the identified condition. The processor 130 may update the dehydration criteria information using the weight corresponding to the identified condition.

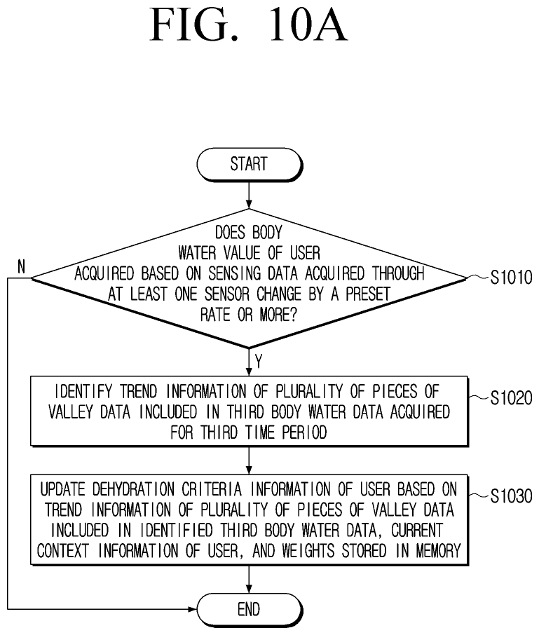

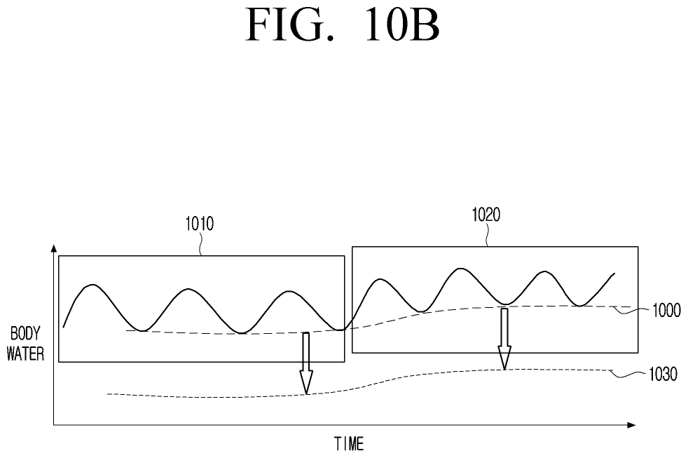

FIGS. 10A and 10B are diagrams for describing a method of updating dehydration criteria information according to an embodiment.

Referring to FIG. 10A, according to an embodiment, the control method may first identify whether the acquired body water value of the user changes by a preset rate or more, based on the sensing data acquired through at least one sensor 110 (S1010).

According to one example, referring to FIG. 10B, the processor 130 may acquire body water data 1010 of a user during the first time period and body water data 1020 of a user during the second time period after the first time period based on the sensing data acquired through at least one sensor 110. The processor 130 may use the body water data 1010 of the user during the first time period and the body water data 1020 of the user during the second time period after the first time period to identify whether the body water value of the user changes by the preset rate or more.

For example, the processor 130 may identify an average value of the body water value of the user during the first time period based on the body water data 1010 of the user during the first time period, and identify an average value of the body water value of the user during the second time period based on the body water data 1020 of the user during the second time period. The processor 130 may compare the average value of the identified body water value of the user during the first time period with the average value of the body water value of the user during the second time period to determine whether the change rate is greater than or equal to the preset rate (e.g., whether the difference is greater than or equal to 5%).

However, the present disclosure is not limited thereto, and for example, the processor 130 may compare the trend information corresponding to the body water value during the first time period with the trend information corresponding to the body water value during the second time period to identify whether the body water value of the user changes by the preset rate or more.

Referring back to FIG. 10A, according to an embodiment, when the control method identifies that the body water value of the user has changed by the preset rate or more (S1010: Y), the control method may identify the trend information of the plurality of pieces of valley data included in third body water data acquired during a third time period (S1020).

According to one example, by comparing the average value of the identified body water value of the user during the first time period with the average value of the body water value of the user during the second time period, the case where the changed rate is identified as being greater than or equal to the preset rate is considered. The processor 130 may acquire the body water data of the user during the third time period after the second time period through at least one sensor. The processor 130 may identify the trend information of the plurality of pieces of valley data included in the acquired third body water data.

Alternatively, according to one example, the processor 130 may identify the trend information of the valley data included in the body water data of the user during the second time period. For example, the processor 130 may compare the average value of the identified body water value of the user during the first time period with the average value of the body water of the user value during the second time period, and identify the trend information based on the body water data of the user during the second time period, when the changed rate is identified to be greater than or equal to the preset rate.

Alternatively, according to one example, the processor 130 may identify the trend information using the body water data of the user during the second time period and the body water data of the user during the third time period. For example, the processor 130 may identify the trend information using the valley data included in the body water data of the user during the second time period and the valley data included in the body water data of the user during the third time period.

Subsequently, according to an embodiment, the control method may update the dehydration criteria information of the user based on the trend information of the plurality of pieces of valley data included in the identified third body water data, the current context information of the user, and the weights stored in the memory (S1030).

According to the above example, when the change occurs in the body water (e.g., weight loss, regular exercise, or taking a diuretic) of the user, the electronic apparatus 100 may identify whether the body water of the user changes by the preset rate or more. When the body water of the user changes by the preset rate or more, the electronic apparatus 100 may identify new user trend information from the time point of change, thereby providing the user with the dehydration criteria information that reflects the change in the body water of the user.

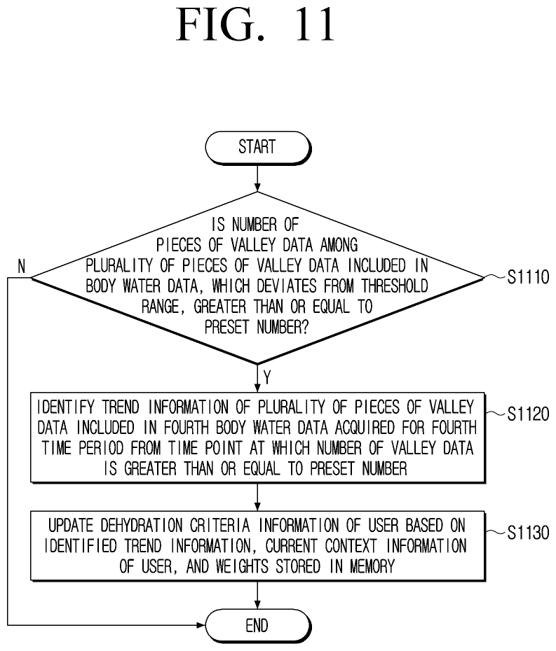

FIG. 11 is a diagram for describing a method of updating dehydration criteria information according to an embodiment.

Referring to FIG. 11, according to an embodiment, the control method may identify whether the number of pieces of valley data included in the body water data that deviates from the threshold range is greater than or equal to the preset number (S1110).

According to an example, the processor 130 may calculate the threshold range based on the identified trend information. For example, the processor 130 may calculate the threshold range based on the value at which the identified trend information decreases by the preset rate (e.g., the value at which the trend information decreases by 3%) and the value at which the trend information increases by the preset rate (e.g., the value at which the trend information increases by 3%). However, the present disclosure is not limited thereto, and it goes without saying that the information on the threshold range may be the value pre-stored in the memory 120 according to an example.

According to an example, when the processor 130 calculates the threshold range based on the value in which the identified trend information decreases by the preset rate (e.g., the value in which the trend information decreases by 3%) and the value in which the trend information increases by the preset rate (e.g., the value in which the trend information increases by 3%), the processor 130 may identify whether the number of data exceeding the calculated critical range is equal to or greater than the preset number.

For example, the processor 130 may identify the number of pieces of valley data exceeding the threshold range among the plurality of pieces of valley data included in the body water data during the preset time period. The processor 130 may identify whether the number of pieces of valley data exceeding the critical range during the preset time period is equal to or greater than the preset number.

Next, according to an embodiment, when the number of pieces of valley data exceeding the threshold range is identified as being greater than or equal to the preset number (S1110: Y), the control method may identify the trend information of the plurality of pieces of valley data included in fourth body water data acquired during a fourth time period from the time point at which the number is identified as being greater than or equal to the preset number (S1120).

Next, according to an embodiment, the control method may update the dehydration criteria information of the user based on the identified trend information, the current context information of the user, and the weights stored in the memory (S1130).

According to the above-described example, when the change in the body water of the user requires the change in the current dehydration criteria information, the electronic apparatus 100 may newly identify the trend information of the user, thereby providing the user with the dehydration criteria information that reflects the change in the body water of the user.

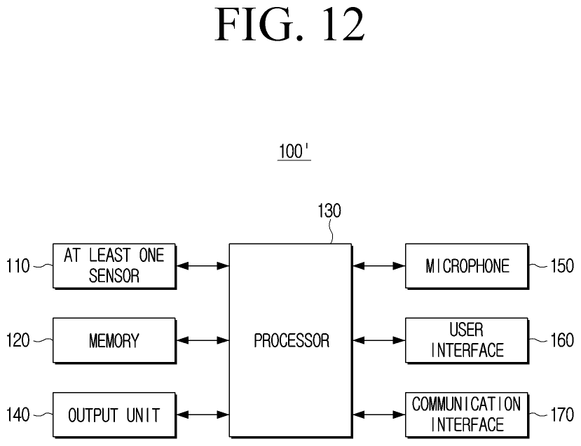

FIG. 12 is a block diagram illustrating a detailed configuration of an electronic apparatus according to an embodiment.

Referring to FIG. 12, an electronic apparatus 100′ may include at least one sensor 110, a memory 120, one or more processors 130 comprising processing circuitry, an output unit 140 comprising a display and/or speaker, a microphone 150, a user interface 160, and a communication interface 170 comprising interface circuitry. A detailed description for components overlapped with components illustrated in FIG. 2 among components illustrated in FIG. 12 will be omitted.

The output unit 140 is a device that outputs various types of information to provide information to a user. For example, the output unit 140 may be implemented as at least one of a speaker 140-1 or a display 140-2, but is not limited thereto.

The speaker 140-1 may include a tweeter for high-pitched sound reproduction, a mid-range sound for mid-range sound reproduction, a woofer for low-pitched sound reproduction, a subwoofer for extremely low-pitched sound reproduction, an enclosure for controlling resonance, a crossover network that divides an electric signal frequency input to the speaker by band, etc.

The speaker 140-1 may output a sound signal to the outside of the electronic apparatus 100′. The speaker 140-1 may output multimedia reproduction, recording reproduction, various kinds of notification sounds, voice messages, and the like. The electronic apparatus 100′ may include an audio output device such as the speaker 140-1, or may include an output device such as an audio output terminal. In particular, the speaker 140-1 may provide acquired information, information processed/produced based on the acquired information, a response result to a user's voice, an operation result, or the like in the form of voice. “Based on” as used herein covers based at least on.

The display 140-2 may be implemented as a display including a self-light emitting element or a display including a non-light emitting element and a backlight. For example, the display 120 may be implemented as various types of displays such as a liquid crystal display (LCD), an organic light emitting diodes (OLED) display, light emitting diodes (LED), a micro LED, a Mini LED, a plasma display panel (PDP), a quantum dot (QD) display, and quantum dot light-emitting diodes (QLED). A driving circuit, a backlight unit, and the like, that may be implemented in a form such as a-si TFT, low temperature poly silicon (LTPS) TFT, an organic TFT (OTFT), and the like, may be included in the display 140-2.

Meanwhile, the display 140-2 may be implemented as a touch screen coupled with a touch sensor, a flexible display, a rollable display, a 3D display, a display to which a plurality of display modules are physically connected, and the like. The processor 130, comprising processing circuitry, may control the display 140-2 to output the output image obtained according to various embodiments described above. Here, the output image may be a high-resolution image of 4K or 8K or higher.

Meanwhile, according to another embodiment, the electronic apparatus 100′ may not include the display 140-2. The electronic apparatus 100′ may be connected to an external display device and may transmit images or content stored in the electronic apparatus 100′ to the external display device. Specifically, the electronic apparatus 100′ may transmit the images or content to the external display device along with a control signal for controlling the display of the images or content on the external display device.

Here, the external display device may be connected to the electronic apparatus 100′ via at least the communication interface 170 or the input/output interface (not illustrated). For example, the electronic apparatus 100′ may not include a display like a set top box (STB). Also, the electronic apparatus 100′ may include only a small display capable of displaying only simple information such as text information. Here, the electronic apparatus 100′ may transmit the images or content to the external display device through the communication interface 170 via a wire or wireless connection, or via an input/output interface (not illustrated).

The microphone 150 may refer to a module that acquires sound and converts the acquired sound into an electrical signal, and may be a condenser microphone, a ribbon microphone, a moving coil microphone, a piezoelectric element microphone, a carbon microphone, or a micro electro mechanical system (MEMS) microphone. In addition, the microphone 150 may be implemented in non-directional, bi-directional, unidirectional, sub cardioid, super cardioid, and hyper cardioid methods.

There may be various embodiments in which the electronic apparatus 100′ performs an operation corresponding to the user voice signal received through the microphone 150.

The user interface 160 is a component for the electronic apparatus 100′ to perform an interaction with a user. For example, the user interface 160 may include at least one of a touch sensor, a motion sensor, a button, a jog dial, a switch, a microphone, or a speaker, but is not limited thereto.

The communication interface 170 may input and output various types of data. For example, the communication interface 170 may transmit and receive various types of data to and from an external device (e.g., source device), an external storage medium (e.g., USB memory), an external server (e.g., web hard), etc., through communication methods such as AP-based Wi-Fi (wireless LAN network), Bluetooth, Zigbee, a wired/wireless local area network (LAN), a wide area network (WAN), Ethernet, IEEE 1394, a high-definition multimedia interface (HDMI), a universal serial bus (UBS), a mobile high-definition link (MHL), an audio engineering society/European broadcasting union (AES/EBU), optical, and coaxial.

According to one example, the communication interface 170 may include a Bluetooth low energy (BLE) module. The BLE refers to Bluetooth technology that enables low-power, low-capacity data transmission and reception in the 2.4 GHz frequency band with a range of approximately 10 meters. However, the present disclosure is not limited thereto, and the communication interface 170 may also include a Wi-Fi communication module. That is, the communication interface 170 may include at least one of the Bluetooth low energy (BLE) module and the Wi-Fi communication module.

According to an example, the communication interface 170 may use different communication modules to communicate with an external device such as a remote control device and an external server. For example, the communication interface 170 may use at least one of the Ethernet module or the WiFi module to communicate with the external server, and may use a Bluetooth module to communicate with the external device such as the remote control device. However, this is only an example, and the communication interface 170 may use at least one of various communication modules in a case in which it communicates with a plurality of external devices or external servers.

According to the above-described example, the electronic apparatus 100′ may acquire the dehydration criteria information of the user by using the body water data of the user, the different types of context information of the user, and the weights corresponding to each of the different types of context information, and provide the information related to whether the user is dehydrated based on the acquired dehydration criteria information. The electronic apparatus 100′ may determine whether the user is dehydrated by reflecting the context information of the user, and thus may provide the information related to whether the user is dehydrated by taking into account the individual characteristics of the user.

Meanwhile, the above-described methods according to various embodiments of the present disclosure may be implemented in a form of application that may be installed in the existing electronic apparatus. Alternatively, the above-described methods according to various embodiments of the present disclosure may be performed using a deep learning-based learned neural network (or deep learned neural network), that is, a learning network model. In addition, the above-described methods according to various embodiments of the present disclosure may be implemented only by software upgrade or hardware upgrade of the existing electronic apparatus. In addition, various embodiments of the present disclosure described above can be performed through an embedded server provided in the electronic apparatus or a server outside the electronic apparatus.

Meanwhile, according to an embodiment of the present disclosure, various embodiments described above may be implemented by software including instructions stored in a machine-readable storage medium (for example, a computer-readable storage medium). A machine may be an apparatus that invokes the stored instruction from the storage medium and may be operated depending on the invoked instruction, and may include the display apparatus (for example, the display apparatus A) according to the disclosed embodiments. In the case in which a command is executed by the processor, the processor may directly perform a function corresponding to the command or other components may perform the function corresponding to the command under a control of the processor. The command may include codes provided or executed by a compiler or an interpreter. The machine-readable storage medium may be provided in a form of a non-transitory storage medium. Here, the term “non-transitory” means that the storage medium is tangible without including a signal, and does not distinguish whether data are semi-permanently or temporarily stored in the storage medium.

In addition, according to an embodiment, the above-described methods according to the diverse embodiments may be included and provided in a computer program product. The computer program product may be traded as a product between a seller and a purchaser. The computer program product may be distributed in a form of a storage medium (for example, a compact disc read only memory (CD-ROM)) that may be read by the machine or online through an application store (for example, PlayStore™). In case of the online distribution, at least a portion of the computer program product may be at least temporarily stored in a storage medium such as a memory of a server of a manufacturer, a server of an application store, or a relay server or be temporarily provided.

In addition, each of components (for example, modules or programs) according to various embodiments described above may include a single entity or a plurality of entities, and some of the corresponding sub-components described above may be omitted or other sub-components may be further included in the diverse embodiments. Alternatively or additionally, some components (e.g., modules or programs) may be integrated into one entity and perform the same or similar functions performed by each corresponding component prior to integration. Operations performed by the modules, the programs, or the other components according to the diverse embodiments may be executed in a sequential manner, a parallel manner, an iterative manner, or a heuristic manner, at least some of the operations may be performed in a different order or be omitted, or other operations may be added.

Although exemplary embodiments of the present disclosure have been illustrated and described hereinabove, the present disclosure is not limited to the abovementioned specific exemplary embodiments, but may be variously modified by those skilled in the art to which the present disclosure pertains without departing from the gist of the present disclosure as disclosed in the accompanying claims. These modifications should also be understood to fall within the scope and spirit of the present disclosure.

1. An electronic apparatus, comprising:
at least one sensor;
a memory in which weights for different types of context information related to a user are to be stored; and
one or more processors, comprising processing circuitry, individually and/or collectively configured to:
in response to first body water data of the user corresponding to a first time period acquired based on sensing data acquired through the at least one sensor, identify trend information of a plurality of pieces of valley data included in the acquired first body water data;
identify dehydration criteria information of the user, based on the trend information, current context information of the user, and weight stored in the memory; and
identify whether the user is dehydrated, based on the body water data acquired through the at least one sensor and the identified dehydration criteria information of the user.

2. The electronic apparatus as claimed in claim 1, wherein, when second body water data of the user corresponding to a second time period after the first time period is acquired, the one or more processors are individually and/or collectively configured to update the trend information based on the first body water data and the second body water data,
update the dehydration criteria information of the user based on the updated trend information, and
identify whether the user is dehydrated after the second time period based on the body water data acquired through the at least one sensor and the updated dehydration criteria information.

3. The electronic apparatus as claimed in claim 1, further comprising:
an output unit comprising a display and/or a speaker,
wherein when it is identified that the body water data acquired through the at least one sensor is less than the identified dehydration criteria information of the user, the one or more processors are individually and/or collectively configured so that the notification information notifying that the user is in the dehydrated state is provided through at least the output unit.

4. The electronic apparatus as claimed in claim 3, wherein the one or more processors are individually and/or collectively configured to provide a graphic user interface (UI) including the notification information through the display and/or provide a sound user interface (UI) corresponding to the notification information through the speaker.

5. The electronic apparatus as claimed in claim 1, wherein the one or more processors are individually and/or collectively configured to identify slopes between the plurality of pieces of valley data included in the body water data and at least one peak data adjacent to each of the plurality of pieces of valley data, respectively, and
when at least one valley data having a slope greater than or equal to a preset value is identified among the identified slopes, identify trend information of the remaining valley data excluding the at least one identified valley data.

6. The electronic apparatus as claimed in claim 5, wherein the one or more processors are individually and/or collectively configured to identify a first slope and a second slope between the plurality of pieces of valley data included in the body water data and first peak data and second peak data adjacent to each of the plurality of pieces of valley data, respectively, and
when at least one valley data is identified in which at least one of the identified first slope and second slope has a slope greater than or equal to the preset value, identify the trend information of the remaining valley data excluding the at least one identified valley data.

7. The electronic apparatus as claimed in claim 1, wherein the memory stores different condition-specific weights corresponding to each of the different types of context information,
the one or more processors are individually and/or collectively configured to identify a weight corresponding to a condition to which each of the at least one pieces of context information related to the user belongs based on the information stored in the memory, and
update the dehydration criteria information of the user based on the identified weights, and
identify whether the user is dehydrated based on the body water data acquired through the at least one sensor and the updated dehydration criteria information of the user.

8. The electronic apparatus as claimed in claim 7, wherein the different types of context information related to the user include at least one of user's weight, height, age, body temperature, exercise frequency, drinking status, and medication information, ambient temperature of the electronic apparatus, and ambient humidity of the electronic apparatus, and
the one or more processors are individually and/or collectively configured to acquire the different types of context information related to the user based on at least one of a user input, sensing data acquired through the at least one sensor, or information received from an external device, and
identify a weight corresponding to a condition to which each of the different types of context information related to the user belongs based on the acquired different types of context information and the different condition-specific weights corresponding to each of the different types of context information stored in the memory.

9. The electronic apparatus as claimed in claim 1, wherein, when the body water value of the user acquired based on sensing data acquired through the at least one sensor changes by a preset rate or more, the one or more processors are individually and/or collectively configured to identify trend information of a plurality of pieces of valley data included in third body water data acquired during a third time period, and
update the dehydration criteria information of the user based on the trend information of the plurality of pieces of valley data included in the identified third body water data, the current context information of the user, and the weights stored in the memory.

10. The electronic apparatus as claimed in claim 1, wherein, when it is identified that the number of pieces of valley data among the plurality of pieces of valley data included in the body water data, which deviates from the threshold range, the one or more processors are individually and/or collectively configured to identify trend information of a plurality of pieces of valley data included in fourth body water data acquired for a fourth time period from a time point at which the number of pieces of valley data is greater than or equal to the preset number, and
update the dehydration criteria information of the user based on the identified trend information, the current context information of the user, and the weights stored in the memory.

11. A method of controlling an electronic apparatus, the method comprising:
in response to first body water data of a user corresponding to a first time period acquired based on sensing data acquired through the at least one sensor, identifying trend information of a plurality of pieces of valley data included in the acquired first body water data;
identifying the dehydration criteria information of the user based on the trend information, current context information of the user, and weights for different types of context information related to the user stored in memory; and
identifying whether the user is dehydrated, based on the body water data acquired through the at least one sensor and the identified dehydration criteria information of the user.

12. The method as claimed in claim 11, further comprising:
in response to second body water data of the user corresponding to a second time period after the first time period being acquired, updating the trend information based on the first body water data and the second body water data;
updating the dehydration criteria information of the user based on the updated trend information; and
identifying whether the user is dehydrated after the second time period based on the body water data acquired through the at least one sensor and the updated dehydration criteria information.

13. The method as claimed in claim 11, further comprising:
when it is identified that the body water data acquired through the at least one sensor is less than the identified dehydration criteria information of the user, providing the notification information notifying that the user is in the dehydrated state through an output unit.

14. The method as claimed in claim 13, wherein, in the providing of the notification information through the output unit, a graphic user interface (UI) including the notification information is provided through a display and/or a sound user interface (UI) corresponding to the notification information is provided through a speaker.

15. A non-transitory computer-readable storage medium storing a computer command that, when executed by a processor of an electronic apparatus, causes the electronic apparatus to perform an operation comprising:
in response to first body water data of the user, corresponding to a first time period, acquired based on sensing data acquired through the at least one sensor, identifying trend information of a plurality of pieces of valley data included in the acquired first body water data;
identifying the dehydration criteria information of the user based on the trend information, current context information of the user, and weights for different types of context information related to the user stored in memory; and
identifying whether the user is dehydrated, based on the body water data acquired through the at least one sensor and the identified dehydration criteria information of the user.

16. The non-transitory computer-readable storage medium as claimed in claim 15, the operation further comprising:
in response to second body water data of the user corresponding to a second time period after the first time period being acquired, updating the trend information based on the first body water data and the second body water data;
updating the dehydration criteria information of the user based on the updated trend information; and
identifying whether the user is dehydrated after the second time period based on the body water data acquired through the at least one sensor and the updated dehydration criteria information.

17. The non-transitory computer-readable storage medium as claimed in claim 15, the operation further comprising:
when it is identified that the body water data acquired through the at least one sensor is less than the identified dehydration criteria information of the user, providing the notification information notifying that the user is in the dehydrated state through an output unit.

18. The non-transitory computer-readable storage medium as claimed in claim 17, wherein, in the providing of the notification information through the output unit, a graphic user interface (UI) including the notification information is provided through a display and/or a sound user interface (UI) corresponding to the notification information is provided through a speaker.

19. The non-transitory computer-readable storage medium as claimed in claim 15, the operation further comprising:
identifying slopes between the plurality of pieces of valley data included in the body water data and at least one peak data adjacent to each of the plurality of pieces of valley data, respectively, and
based on at least one valley data having a slope greater than or equal to a preset value being identified among the identified slopes, identifying trend information of the remaining valley data excluding the at least one identified valley data.

20. The non-transitory computer-readable storage medium as claimed in claim 19, the operation further comprising:
identifying a first slope and a second slope between the plurality of pieces of valley data included in the body water data and first peak data and second peak data adjacent to each of the plurality of pieces of valley data, respectively, and
based on at least one valley data being identified in which at least one of the identified first slope and second slope has a slope greater than or equal to the preset value, identifying the trend information of the remaining valley data excluding the at least one identified valley data.
